(19) United States
(12) Patent Application Publication
(10) Pub. No.: US 2021/0007954 A1
(43) Pub. Date: Jan. 14, 2021

(54) SOLID HAIR COSMETIC COMPOSITION

(71) Applicant: Henkel AG & Co. KGaA, Duesseldorf (DE)

(72) Inventors: SOEREN SCHEELE, Pinneberg (DE); MANUELA METTE, Kleinfeld (DE); PETRA WESTPHAL, Neu Wulmstorf (DE); THOMAS SCHROEDER, Hamburg (DE)

(73) Assignee: Henkel AG & Co. KGaA, Duesseldorf (DE)

(21) Appl. No.: 16/926,392

(22) Filed: Jul. 10, 2020

(30) Foreign Application Priority Data
Jul. 10, 2019 (DE) 10 2019 210 154.2

(51) Int. Cl. A61K 8/34; A61K 8/73; A61K 8/02; A61Q 5/02

(52) U.S. Cl. CPC A61K 8/345; A61K 8/732; A61K 8/738; A61K 2800/594; A61Q 5/02; A61K 2800/5424; A61K 8/0229

(57) ABSTRACT

A solid cosmetic composition comprising—based on the total weight of the cosmetic composition—from about 30.0 to about 60.0% by weight of at least one polyhydric alcohol, and optionally: from about 15.0 to about 50.0% by weight of at least one surfactant, from about 0.1 to about 15.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and from about 0.1 to about 20.0% by weight of at least one polysaccharide, as well as production and application methods and uses thereof.

### CROSS-REFERENCE TO RELATED APPLICATION

This application claims priority to German Patent Application No. 10 2019 210 154.2, filed Jul. 10, 2019, which is incorporated herein by reference in its entirety.

### TECHNICAL FIELD

The notification describes solid cosmetic compositions based on higher amounts of polyols, in particular solid cleansing compositions which dissolve and foam in contact with water. The solid cosmetic compositions possibly other ingredients such as surfactants, polysaccharides, fatty alcohols and/or fatty acid(s). The notification further describes processes for the preparation of solid cosmetic compositions and their use for the cleansing of the human body, including the hair.

### BACKGROUND

Surfactant-containing products for cleansing the human body and hair have been known for a long time and are mainly sold as foam baths, shower preparations, shampoos and/or oral care compositions in liquid or paste form in suitable packaging. End users take the required amount of product from the packaging during use and dispose of it after emptying. Compared to classic soaps that are sold in solid form, such products offer the user the advantage of easy and quick handling, which is why they dominate the market today. However, this advantage is achieved by accepting certain disadvantages, which are discussed below. In most cases, the packaging of the described products in liquid or paste form includes non-recyclable plastic, which is a serious problem from an environmental point of view in view of the constantly growing amount of plastic waste.

Another problem is that previous products usually contain higher quantities of water or water/solvent mixtures, which means that the products have a larger volume and, possibly of greater importance from a transport point of view, a relatively high weight. This is disadvantageous for several reasons. In times of increasing water scarcity, resources should be saved. An undesirable, increased transport volume associated with large-volume heavy products is also important from an environmental and cost perspective. Another interesting point is that worldwide travel activity is constantly increasing. Consumers are therefore increasingly interested in cosmetic products that are easy to transport due to their low weight and volume. This is particularly relevant with regard to air travel, as larger containers containing liquids are generally excluded from being carried in an aircraft cabin, so that a passenger travelling only with hand luggage often finds himself in the situation, due to the cosmetics products that dominate the market today, of not being able to take his preferred product selection with him or having to decant the corresponding products into smaller containers first, which, however, is generally accompanied by an even greater volume of packaging material.

The provision of alternative product forms with lower water content, which are contained in more environmentally friendly, for example recyclable, packaging to save space, is therefore an important goal in the formulation of improved, contemporary and sustainable cosmetic products.

Classic bar soaps have long been known and used. They also generally have a very low water or solvent content and are often packed with little material but their handling is not pleasant for many people because a started soap bar is difficult to transport, often partially dissolves when lying next to the sink, which is also inefficient, and makes the sink look unattractive due to soap residues and because soap bars have a tendency to slip out of the user's hand when wet.

Furthermore, classic soap formulations are only conditionally suitable for the regular cleaning of the entire human body. Due to their skin dehydration potential, they are mainly used for cleaning hands, which are more tolerant in this respect than other skin areas. Furthermore, bar soaps are controversial from a hygiene point of view because of their multiple uses, in particular when they are used by several final consumers.

Another disadvantage of conventional soap formulation, especially with rather small bars of soap, is that it takes some time for enough of the soap bar to come off to achieve the desired amount of lather and the desired cleaning effect. On the one hand, this is usually not desired by users because of the additional time required, and on the other hand it can be associated with higher water consumption for personal hygiene, as many users do not turn off the water flow of the shower or tap when soaping. From these points of view, it does not make sense to market single application portions of classic soap formulations, especially in miniaturized form of a classic soap bar, as their dissolution is too slow as the formulations of these soaps are not optimized for cleaning larger skin areas or hair.

### BRIEF SUMMARY

Solid hair cosmetic compositions, processes for preparing the same, and processes for treating hair using the same are provided herein. In an embodiment, a solid hair cosmetic composition includes—based on the total weight of the cosmetic composition—

a) from about 30.0 to about 60.0% by weight of at least one polyhydric alcohol, and optionally:

b) from about 15.0 to about 50.0% by weight of at least one surfactant,

c) from about 0.1 to about 15.0.% by weight of at least one saturated or unsaturated, branched or unbranched C₈-C₃₀ alcohol and/or a saturated or unsaturated, branched or un-branched C₈-C₃₀ carboxylic acid and/or a salt of a saturated or unsaturated, branched or un-branched C₈-C₃₀ carboxylic acid, and

from about 0.1 to about 20.0% by weight of at least one polysaccharide.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In view of the problems and requirements described above, formulations and manufacturing processes suitable for solid cosmetic detergents are provided which, by their nature, can be packaged in individual application portions, as well as processes for their manufacture and uses. Several measures have been established that can contribute to this suitability. Thus, the task of the present disclosure is solved by the formulation of procedures and uses described in detail below:

The present disclosure is exemplified by:
1. A solid hair cosmetic composition comprising—based on the total weight of the cosmetic composition—
   a. From about 30.0 to about 60.0% by weight of at least one polyhydric alcohol, and optionally:
   b. from about 15.0 to about 50.0% by weight of at least one surfactant,
   c. from about 0.1 to about 15.0.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
   d. from about 0.1 to about 20.0% by weight of at least one polysaccharide.
2. A solid hair cosmetic composition according to point 1, comprising—based on the total weight of the cosmetic composition—
   a. From about 30.0 to about 60.0% by weight of at least one polyhydric alcohol,
   b. from about 15.0 to about 50.0% by weight of at least one surfactant,
   c. from about 0.1 to about 15.0.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
   d. from about 0.1 to about 20.0% by weight of at least one polysaccharide.
3. A solid hair cosmetic composition according to any of the preceding points, comprising—based on the total weight of the cosmetic composition):
   (ai) from more than from about 30.0% to about 60.0% by weight of at least one polyhydric alcohol.
4. A solid hair cosmetic composition according to any of the preceding points, comprising—based on the total weight of the cosmetic composition):
   ai) from about 32.0 to about 50.0% by weight of at least one polyhydric alcohol.
5. A solid hair cosmetic composition according to any of the preceding points, comprising—based on the total weight of the cosmetic composition—:
   ai) from about 32.0 to about 40.0% by weight of at least one polyhydric alcohol.
6. A solid hair cosmetic composition according to any of the foregoing, comprising or consisting of glycerol as polyhydric alcohol a).
7. A solid hair cosmetic composition according to any of the foregoing, comprising at least one anionic surfactant.
8. A solid hair cosmetic composition according to any of the foregoing, comprising
   i. at least one anionic isethionate surfactant of formula (I)

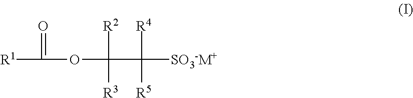

located in the
   R¹ represents a linear or branched, saturated or unsaturated alkyl radical containing 6 to 30 carbon atoms,
   R² to R⁵ independently of one another represent a hydrogen atom, a C₁-C₄ alkyl radical or a C₁-C₄ hydroxyalkyl radical, and
   M⁺ represents an ammonium, an alkanol ammonium or a metal cation and/or
ii. at least one anionic taurate surfactant of formula (II)

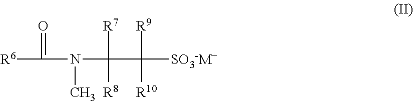

located in the
   R⁶ represents a linear or branched, saturated or unsaturated alkyl radical containing 6 to 30 carbon atoms,
   R⁷ to R¹⁰ independently of one another represent a hydrogen atom, a C₁-C₄ alkyl radical or a C₁-C₄ hydroxyalkyl radical, and
   M⁺ stands for an ammonium, an alkanol ammonium or a metal cation
9. A solid hair cosmetic composition according to point 8, comprising (based on the total weight of the cosmetic composition)
   bi) from about 10.0 to about 40% by weight of at least one anionic isethionate surfactant of formula (I) and
   bii) from about 1.0 to about 10% by weight of at least one anionic taurate surfactant of formula (II).
10. A solid hair cosmetic composition according to point 8 or 9, comprising (based on the total weight of the cosmetic composition)
   ci) from about 20.0 to about 30% by weight of at least one anionic isethionate surfactant of formula (I) and
   cii) from about 1.0 to about 5% by weight of at least one anionic taurate surfactant of formula (II).
11. A solid hair cosmetic composition according to any of the preceding points, containing as component c) saturated or unsaturated, branched or unbranched C₈-C₃₀ carboxylic acids and/or salts thereof, preferably C₁₀-C₂₂ carboxylic acids and/or salts thereof.
12. A solid hair cosmetic composition according to one of the preceding points, containing as component c) coco acids, lauric acid, myristic acid, palmitic acid, stearic acid, behenic acid, oleic acid and mixtures thereof and/or the salts of these acids.
13. A solid hair cosmetic composition according to one of the preceding points, containing as component c) lauric acid, myristic acid, palmitic acid, stearic acid, as well as mixtures thereof and/or the salts of these acids; in particular palmitic acid, stearic acid, as well as mixtures thereof and/or the salts of these acids.
14. A solid hair cosmetic composition according to one of the preceding points, containing as component c) sodium palmitate and/or sodium stearate, in particular sodium palmitate and sodium stearate.
15. A solid hair cosmetic composition according to any of the preceding points, sending as polysaccharide d) at least one starch and/or at least modified starch and/or at least one dextrin.
16. A solid hair cosmetic composition according to any of the foregoing, wherein the polysaccharide d) contains a corn starch.
17. A solid hair cosmetic composition according to any of the foregoing, wherein the polysaccharide d) contains a dextrin.
18. A solid hair cosmetic composition as defined in point 17, where the dextrin is maltodextrin.
19. A solid hair cosmetic composition according to any of the foregoing, wherein the polysaccharide d) contains or consists of a corn starch and maltodextrin.
20. A solid hair cosmetic composition according to any of the foregoing, wherein the polysaccharide d) a maize starch consists thereof.
21. A solid hair cosmetic composition according to any of the foregoing, where the maize starch is Zea Mays (corn) starch.
22. A solid hair cosmetic composition according to any of the foregoing, comprising from about 0.5 to about 15%, preferably from about 2 to about 15% and more preferably from about 5 to about 12% by weight of component c) (based on the total weight of the cosmetic composition).
23. A solid hair cosmetic composition according to any of the foregoing, further comprising, based on the total weight of the cosmetic composition, from about 0.01 to about 5.00% by weight of at least one cationic polymer.
24. A solid hair cosmetic composition according to any of the preceding points, further comprising—based on the total weight of the cosmetic composition—from about 0.1 to about 5.00% by weight, preferably from about 0.1 to about 3% by weight and more preferably from about 0.1 to about 1% by weight of at least one cationic polymer.
25. A solid hair cosmetic composition according to point 23 or 24, comprising as cationic polymer at least one cationic polysaccharide polymer obtainable from guar, cassia and/or inulin.
26. A solid hair cosmetic composition according to any one of points 23 to 25 comprising as cationic polymer hydroxypropyltrimonium inulin.
27. A solid hair cosmetic composition according to any of the foregoing, further comprising—based on the total weight of the cosmetic composition—from about 0.001 to about 0.01% by weight of at least one bittering agent, preferably a compound known under the INCI designation denatonium benzoate.
28. A solid hair cosmetic composition according to any of the preceding points, further comprising—based on the total weight of the cosmetic composition—from about 0.01 to about 10.0% by weight of at least one oil, fat and/or wax component, preferably a naturally occurring oil, fat or wax.
29. A solid hair cosmetic composition according to point 28, containing vegetable oils and/or vegetable butters,
30. A solid hair cosmetic composition according to one of the items 28 or 29, containing Shea Butter (INCI designation: Butyrospermum Parkii (Shea) Butter).
31. A solid hair cosmetic composition according to any one of items 28 to 30, comprising apricot kernel oil, argan oil, jojoba oil, manila oil, almond oil, olive oil, coconut oil and/or sunflower oil.
32. A solid hair cosmetic composition according to any of the foregoing, further comprising citric acid, lactic acid, malic acid and/or glycolic acid, especially citric acid and/or lactic acid.
33. A solid hair cosmetic composition according to any of the foregoing, further comprising sodium bicarbonate.
34. A solid hair cosmetic composition according to any of the foregoing comprising water in an amount up to about 25% by weight (based on the weight of the composition).
35. A solid hair cosmetic composition according to one of the previous points in the form of a pen or piece.
36. A solid hair cosmetic composition according to point 35 in the form of a stick.
37. A solid hair cosmetic composition as defined in point 36, with a Shore AO hardness value of from about 5 to about 20.
38. A solid hair cosmetic composition as defined in point 37, with a Shore AO hardness value of from about 5 to about 15.
39. A solid hair cosmetic composition according to any one of points 36 to 38 comprising water in an amount up to about 25% by weight, preferably about 20% and more preferably about 15% by weight (based on the weight of the composition).
40. A solid hair cosmetic composition according to point 35 in the form of a multiple use piece.
41. A solid hair cosmetic composition as defined in point 35 in the form of a single use piece.
42. A solid hair cosmetic composition as defined in point 41, with a Shore AO hardness value of from about 15 to about 35.
43. A solid hair cosmetic composition as defined in point 42, with a Shore AO hardness value of from about 20 to about 30.
44. A solid hair cosmetic composition according to any one of points 41 to 43 comprising water in an amount up to about 15% by weight, preferably about 12.5% and more preferably about 10% by weight (based on the weight of the composition).
45. A solid hair cosmetic composition according to any of the preceding points for the cleansing and/or care of human skin and/or human hair.
46. A solid hair cosmetic composition according to any of the previous points in the form of a detergent.
47. A process for preparing the solid hair cosmetic composition according to any of the foregoing, comprising the process:
   a. Mix all ingredients with sufficient heat and agitation to ensure that all ingredients are melted and/or evenly distributed,
   b. Allow the resulting mixture to cool and solidify.
48. A process for preparing the solid hair cosmetic composition according to any one of points 1 to 46, comprising the process:
   a. Mix all ingredients with sufficient heat and agitation to ensure that all ingredients are melted and/or evenly distributed,
   b. Pour the resulting mixture into a pin/piece mould,
   c. Allow the mixture to cool and solidify.
49. A process for preparing the solid hair cosmetic composition according to any one of points 1 to 46, comprising the process:
   a. Mix all ingredients with sufficient heat and agitation to ensure that all ingredients are melted and/or evenly distributed,
   b. Pour the resulting mixture into a mould,
   c. Allow the mixture to cool and solidify.
50. A method for cleansing hair in which a solid hair cosmetic composition is mixed with water according to any one of points 1 to 46 and applied to the hair or in which a pen or piece is applied directly to wet hair and a working-in is performed.
51. A solid hair cosmetic composition prepared by a method according to one of the definitions of points 47 to 50.
52. The use of a solid hair cosmetic composition according to any one of points 1 to 46 for cleansing and/or conditioning hair.

The present disclosure thus concerns a solid hair cosmetic composition comprising—based on the total weight of the cosmetic composition—
a) from about 30.0 to about 60.0% by weight of at least one polyhydric alcohol, and optionally:
b) from about 15.0 to about 50.0% by weight of at least one surfactant,
c) from about 0.1 to about 15.0.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20.0% by weight of at least one polysaccharide.

Such a formulation offers the right properties for single application portions, especially with regard to their dissolving and foaming behaviour during use.

The present disclosure further relates to a solid hair cosmetic composition as described above, comprising—based on the total weight of the cosmetic composition—
a) from about 30.0 to about 60.0% by weight of at least one polyhydric alcohol,
b) from about 15.0 to about 50.0% by weight of at least one surfactant,
c) from about 0.1 to about 15.0.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20.0% by weight of at least one polysaccharide.

The compositions as contemplated herein are solid at 25° C. Solid compositions within the meaning of the present application are three-dimensional, dimensionally stable entities which are not liquid or gaseous, that is to say, which retain their external shape even without a surrounding vessel. However, the term “solid” does not imply anything about density or elasticity or other physical properties, so that jellies, brawn, butter etc. can also be solid as contemplated herein as long as they are dimensionally stable at 25° C.

Such a formulation offers the right properties for single application portions, especially with regard to their dissolving and foaming behaviour during use. The high concentrations of the active substances in such a composition are associated with the advantages that few resources are consumed during production and transport and that the products, even after they have reached the hands of a consumer via the trade, can be easily transported without great effort or restrictions, whether to the gym or on a flight.

The present disclosure further relates to a solid hair cosmetic composition as described above, comprising (based on the total weight of the cosmetic composition):
ai) more than from about 30.0 to about 60.0% by weight of at least one polyhydric alcohol.

Polyhydric alcohols are mostly well tolerated by the skin and as solvents they ensure that the solid cosmetic compositions available with them are not too solid or too difficult or slow to dissolve. In the defined concentration range, these advantageous properties become particularly apparent.

The present disclosure also relates to a solid hair cosmetic composition as described above, containing as polyhydric alcohol a) alditols such as mannitol, isomalt, lactitol, sorbitol and xylitol, threit, erythritol and arabitol, 1,2-propylene glycol, 1,3-butylene glycol, dipropylene glycol, glycerol and/or diglycerol, preferably glycerol. As contemplated herein, compositions preferably contain glycerol in the quantities mentioned above.

The present disclosure further relates to a solid hair cosmetic composition as previously described, comprising or consisting of glycerol as polyhydric alcohol a).

The present disclosure further relates to a solid hair cosmetic composition as described above, comprising (based on the total weight of the cosmetic composition):
ai) from about 32.0 to about 50.0% by weight of at least one polyhydric alcohol.

In the defined concentration range, the advantageous properties described above become even more apparent.

The present disclosure further relates to a solid hair cosmetic composition as described above, comprising (based on the total weight of the cosmetic composition):
b) from about 15.0 to about 50.0% by weight of at least one surfactant.

The solid hair cosmetic composition described above may contain an anionic, amphoteric, zwitterionic or non-ionic surfactant or a mixture thereof.

The present disclosure further relates to a solid hair cosmetic composition as described above, comprising at least one anionic surfactant.

Anionic surfactants carry a negative charge in their hydrophilic part. Chemically, these are usually salts of sodium, potassium, ammonium, magnesium, monoethanolamine, diethanolamine or triethanolamine.

Suitable anionic surfactant types which can be used in the agents as contemplated herein include
linear and branched fatty acids with from about 8 to about 30 C atoms (Soaps),
Ethercarboxylic acids of the formula R—O—(CH₂—CH₂O)ₓ—CH₂—COOH, in which R is a linear or branched, saturated or unsaturated alkyl group having from about 8 to about 30 C atoms and x=0 or 1 to 16
Acylsarcosides with from about 8 to about 24 C atoms in the acyl group (sarcosinate surfactants)
Acyltaurides with from about 8 to about 24 C atoms in the acyl group (taurate surfactants),
Acyl isethionates with from about 8 to about 24 C atoms in the acyl group (isethionate surfactants),
Sulphosuccinic acid mono- and/or dialkyl esters with from about 8 to about 24 C atoms in the alkyl group and Sulphosuccinic acid mono-alkyl polyoxyethyl esters with from about 8 to about 24 C atoms in the alkyl group and from about 1 to about 6 oxyethyl groups (sulphosuccinate surfactants),
Alpha-olefin sulphonates with from about 8 to about 24 C atoms (alpha-olefin sulphonate surfactants),
Alkyl sulphates and/or alkyl ether sulphate salts of the formula R—(OCH₂—CH₂)ₙ—O—SO₃X, in which R is preferably a straight-chain or branched, saturated or unsaturated alkyl group containing from about 8 to about 30 carbon atoms, x is the number 0 or 1 to 12 and X is an alkali metal, alkaline earth metal, ammonium or alkanolamine ion,
Sulfonates of unsaturated fatty acids with from about 8 to about 24 C atoms and 1 to 6 double bonds,
Esters of tartaric acid and citric acid with alcohols which are addition products of from about 2-15 molecules of ethylene oxide and/or propylene oxide to fatty alcohols with from about 8 to about 22 C atoms, and/or
Alkyl and/or alkenyl ether phosphates of the formula

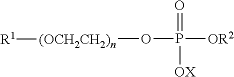

in which R¹ is preferably an aliphatic hydrocarbon radical having from about 8 to about 30 carbon atoms, R² is hydrogen, a radical (CH₂CH₂O)ₙR¹ or X, n is numbers from 0 to 10 and X is hydrogen, an alkali metal or alkaline earth metal or the group —NR³R⁴R⁵R⁶, where R³ to R⁶ independently of one another are a C₁ to C₄ hydrocarbon radical.

Amino acid-based surfactants are particularly suitable in the sense of the present disclosure because they exhibit excellent skin compatibility and they are available from natural sources.

Due to their particularly good incorporation into solid compositions within the meaning of the present disclosure, isethionate and/or taurate surfactants have proved to be particularly suitable.

The present disclosure further relates to a solid hair cosmetic composition as described above, comprehensive
iii. at least one anionic isethionate surfactant of formula (I)

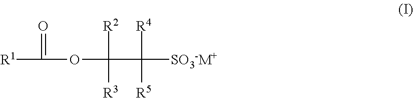

located in the
R¹ represents a linear or branched, saturated or unsaturated alkyl radical containing from about 6 to about 30 carbon atoms,
R² to R⁵ independently of one another represent a hydrogen atom, a C₁-C₄ alkyl radical or a C₁-C₄ hydroxyalkyl radical, and
M⁺ represents an ammonium, an alkanol ammonium or a metal cation and/or
iv. at least one anionic taurate surfactant of formula (II)

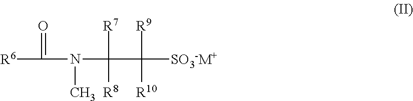

located in the
R⁶ represents a linear or branched, saturated or unsaturated alkyl radical containing from about 6 to about 30 carbon atoms,
R⁷ to R¹⁰ independently of one another represent a hydrogen atom, a C₁-C₄ alkyl radical or a C₁-C₄ hydroxyalkyl radical, and
M⁺ stands for an ammonium, an alkanol ammonium or a metal cation

Anionic isethionate and taurate surfactants are mild, anionic surfactants, ideal for the production of shampoos, shampoo bars, body washes, liquid soaps, face care and bath bombs. They make hair easy to comb, smooth, soft and shiny. They can be produced on a purely vegetable basis and can therefore be used for vegan cosmetics. A source of raw materials can be fatty acids from coconut oil.

In a particularly preferred form, the compositions as contemplated herein contain at least one anionic isethionate surfactant according to formula (I) and at least one anionic taurate surfactant according to formula (II).

The present disclosure further relates to a solid hair cosmetic composition as described above, comprising (based on the total weight of the cosmetic composition)
bi) from about 10.0 to about 40% by weight of at least one anionic isethionate surfactant of formula (I) and
bii) from about 1.0 to about 10% by weight of at least one anionic taurate surfactant of formula (II).

These concentration ranges have proven to be particularly suitable. When working with these concentration ranges, the solid cosmetic compositions achievable with them have balanced properties in terms of firmness, solubility behaviour, application feel and cleaning performance

Suitable amphoteric and/or zwitterionic surfactant types which may be used in the cosmetic compositions as contemplated herein include, for example, one or more compounds of the following formulae (i) to (vii). In which the radical R preferably represents a straight-chain or branched, saturated or mono- or polyunsaturated alkyl or alkenyl radical having from about 7 to about 23 carbon atoms (formulae (i) and (ii)) or a straight-chain or branched, saturated or mono- or polyunsaturated alkyl or alkenyl radical having from about 8 to about 24 carbon atoms (formulae (iii) to (vii)):

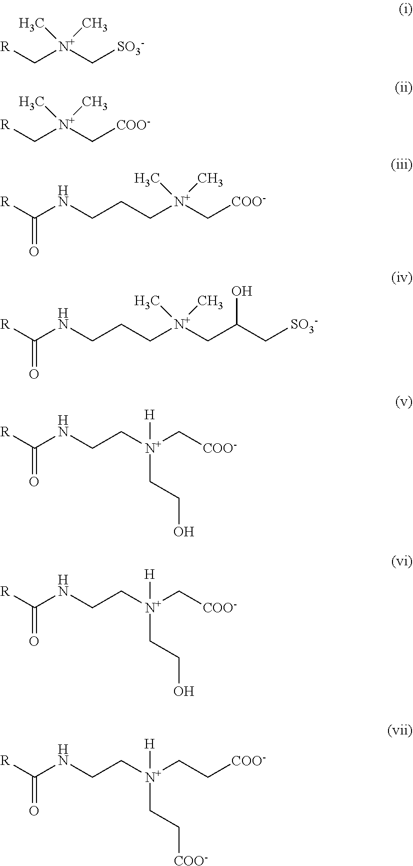

Preferred amphoteric surfactants of the formulae (i) to (vii) mentioned above contain as residue R predominantly a straight-chain or branched, saturated, mono- or polyunsaturated alkyl residue with from about 8 to about 20, more preferably from about 8 to about 18 and in particular with from about 8 to about 16 C atoms. Particularly preferred are amphoteric surfactants, where the rest R is derived from coconut oil.

Particularly preferred are those under the INCI designations Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine Cocamidopropylbetaine and/or Lauramidopropylbetaine known and commercially available amphoteric surfactants from several suppliers.

Surfactants with the INCI designations cocamidopropyl betaine, lauramidopropyl betaine, cocoampho(di)acetates and/or lauroampho(di)acetates are particularly preferred.

The amphoteric and/or zwitterionic co-surfactant(s) is (are) used in the cosmetic compositions as contemplated herein (based on their total weight) preferably in an amount of from about 0 to about 10.00% by weight, more preferably from about 0.1 to about 8.00% by weight, particularly preferably from about 0.5 to about 6.00% by weight and in particular from about 0.75 to about 5.00% by weight.

Suitable non-ionic surfactants for use as co-surfactants include
Amine oxides, which can be selected from compounds of the general formulas (I) or (II)

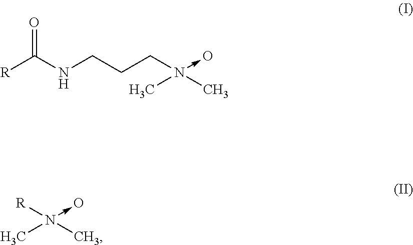

in which each R represents a straight-chain or branched, saturated or mono- or polyunsaturated alkyl or alkenyl radical having from about 6 to about 24 carbon atoms, preferably from about 8 to about 18 carbon atoms.
Particularly preferred are the surfactants of the above-mentioned formula (I) or (II) known under the INCI designations Cocamine Oxide, Lauramine Oxide and/or Cocamidopropylamine Oxide and commercially available from various suppliers.
   Fatty acid alkanolamides of the following general formula,

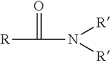

in which R preferably denotes a linear or branched, saturated or unsaturated alkyl or alkenyl radical having from about 8 to about 24 carbon atoms and the radicals R represent hydrogen or the group —(CH₂)ₙOH, in which n denotes the numbers 2 or 3, with the proviso that at least one of the radicals R represents the above mentioned radical —(CH₂)ₙOH,
Mixtures of alkyl (oligo)-glucosides and fatty alcohols for example the commercially available product Montanov®68,
Sterols. Sterols are a group of steroids that carry a hydroxyl group on C atom 3 of the steroid skeleton and are isolated from animal tissue (zoosterols) as well as from vegetable fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
Phospholipids. Among these are mainly the glucose-phospholipids, which are obtained as lecithins or phosphatidylcholines from e.g. egg yolks or plant seeds (e.g. soybeans),
Alkyl(oligo)glycoside. Suitable alkyl (oligo)glycosides can be selected from compounds of the general formula of RO—[G]x, in which [G] is preferably derived from aldoses and/or ketosis with 5-6 carbon atoms, preferably from glucose.
The index number x stands for the degree of oligomerization (DP), i.e. the distribution of mono- and oligoglycosides. The index number x preferably has a value in the range from about 1 to about 10, particularly preferably in the range from about 1 to about 3, whereby it may not be an integer but a fractional number that can be determined analytically.
Particularly preferred alkyl (oligo)glycosides have a degree of oligomerization between about 1.2 and about 1.5. The radical R preferably represents at least one alkyl and/or alkenyl radical containing from about 4 to about 24 carbon atoms.
Especially preferred alkyl (oligo)glycosides are the compounds known under the INCI designations Caprylyl/Capryl Glucosides, Decyl Glucosides, Lauryl Glucosides and Coco Glucosides.

Also fatty alcohol alkoxylates (especially ethoxylates) with a C-chain length of from about 8 to about 24, especially from about 10 to about 20, and degree of alkoxylation (degree of ethoxylation) of from about 2 to about 30 such as Laureth-4 etc. are suitable as contemplated herein.

Particularly preferred non-ionic surfactants, which can be contained in the agents as contemplated herein as co-surfactants, are fatty acid alkanolamides, alkyl (oligo)glucosides and amine oxides. Especially preferred are fatty acid alkanolamides.

The non-ionic co-surfactant(s) is (are) used in the cosmetic compositions as contemplated herein (based on their total weight) preferably in an amount of from about 0 to about 3.00% by weight, more preferably from about 0.10 to about 2.50% by weight, particularly preferably from about 0.20 to about 2.25% by weight and especially from about 0.30 to about 2.00% by weight.

The compositions as contemplated herein can also be conditioning agents in addition to cleaning agents. Preferred conditioning agents as contemplated herein can preferably
contain at least one cationic surfactant, preferably in a total amount of from about 0.1 to about 10% by weight, more preferably from about 0.2 to about 9% by weight and particularly preferably from about 0.3 to about 8% by weight, each based on the weight of the composition, and
at least one further surfactant selected from anionic, amphoteric, zwitterionic and/or non-ionic surfactants, preferably in a total amount of from about 0.1 to about 20% by weight, more preferably from about 0.2 to about 15% by weight and particularly preferably from about 0.3 to about 10% by weight, each based on the weight of the composition.

The present disclosure further relates to a solid hair cosmetic composition as described above, containing as component c) saturated or unsaturated, branched or unbranched C₈-C₃₀ carboxylic acids and/or their salts preferably C₁₀-C₂₂ carboxylic acids and/or their salts and in particular coco acids, lauric acid, myristic acid, palmitic acid, stearic acid, behenic acid, oleic acid as well as mixtures thereof and/or the salts of these acids in the amounts described above.

Particularly suitable are coco acids, stearic acid, palmitic acid and/or the salts of these acids, especially coco acids, sodium stearate and sodium palmitate.

These compounds have proved to be particularly suitable structure-giving ingredients for the purposes of the invention. They can be used to formulate hair cosmetic compositions of sufficient strength that do not melt too low.

In a preferred embodiment, the compositions as contemplated herein contain—based on their total weight—from about 0.5 to about 15% by weight, preferably from about 1.0 to about 10% by weight, particularly preferably from about 1.5 to about 10% by weight and in particular from about 2.0 to about 8% by weight of at least one component c).

The present disclosure further relates to a solid hair cosmetic composition as described above, containing as component d) from about 0.1 to about 20.0% by weight of at least one polysaccharide.

Preferably comprising as polysaccharide d) starch and/or modified starch and/or dextrins.

Polysaccharides suitable for the present disclosure d) are usually composed of more than ten monosaccharide units. Preferred polysaccharides are the starches composed of α-D-glucose units and starch degradation products such as amylose, amylopectin and dextrins. As contemplated herein, chemically and/or thermally modified starches are particularly advantageous, e.g. hydroxypropyl starch phosphate, dihydroxypropyl distarch phosphate or the commercial products Dry Flo®. Dextranes and their derivatives, e.g. dextran sulphate, are also preferred. Non-ionic cellulose derivatives such as methyl cellulose, hydroxypropyl cellulose, hydroxypropyl methyl cellulose or hydroxyethyl cellulose are also preferred. Other preferred examples are polysaccharides from fucose units, such as the commercial product Fucogel®.

The present disclosure also concerns a solid hair cosmetic composition as described above, containing as polysaccharide d)
i. Starch fractions from corn, potatoes, rice, wheat and/or tapioca, and/or derivatives of starches such as amylose, amylopectin, dextrin, and/or
ii. Cellulose and/or cellulose derivatives such as alkyl cellulose, carboxyalkyl cellulose, hydroxyalkyl cellulose

Especially suitable are corn starch and/or maltodextrins, especially corn starch and maltodextrin.

These polysaccharides have proved to be particularly suitable stabilising agents in the context of the present disclosure claimed here. Their use makes it possible to provide ready-made consumer products that retain their properties and appearance over a long period of time and under various environmental conditions.

The present disclosure further relates to a solid hair cosmetic composition as previously described, further comprising—based on the total weight of the cosmetic composition—from about 0.01 to about 5.00% by weight of at least one cationic polymer, preferably at least one cationic polysaccharide polymer obtainable from guar, cassia and/or inulin.

Cationic polymers have conditioning properties, i.e. they provide a pleasant skin or hair feel and thus offer added value. They can be used in the context of the present disclosure without significantly affecting the cleaning performance The specifically named polymers are particularly suitable.

In more detail, suitable cationic care polymers are also to be understood, for example:
quaternized cellulose polymers, especially polyquaternium-10, as commercially available under the names Celquat® and Polymer JR®,
hydrophobically modified cellulose derivatives, for example the cationic polymers sold under the trade name SoftCat®,
cationic alkyl polyglycosides,
cationized honey, for example the commercial product Honeyquat® 50,
cationic guar derivatives, in particular those marketed under the trade names Cosmedia Guar, N-Hance® and Jaguar® distributed products,
polymeric dimethyldiallylammonium salts and their copolymers with esters and amides of acrylic acid and methacrylic acid, especially polyquaternium-6 and polyquaternium-7. The products commercially available under the names Merquat° 100 (poly(dimethyldiallylammonium chloride)) and Merquat®550 (Dimethyldiallylammonium chloride-acrylamide copolymer) are examples of such cationic polymers,
Copolymers of vinylpyrrolidone with quaternized derivatives of dialkylaminoalkyl acrylate and -methacrylate, such as those quaternized with diethyl sulphate Vinylpyrrolidon-Dimethylaminoethylmethacrylat-Copolymere. Such compounds are commercially available under the names Gafquat®734 and Gafquat®755,
Vinylpyrrolidone-vinylimidazolium methochloride copolymers, as offered under the names Luviquat® FC 370, FC 550, FC 905 and HM 552,
quaternized polyvinyl alcohol,
and the products listed under the designations
Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89 known polymers.

Cationic polysaccharide polymers are particularly preferred in the sense of the present disclosure,
which are obtained from natural sources (in particular from guar, cassia and/or inulin)

A content of these specific polymer types in the mixture of active ingredients as contemplated herein is not only beneficial for improving the care properties of skin and hair, but it was also found that polymers in combination with other cationic polymers do not cause an over conditioning effect even after regular application on hair.

Inulin is a polysaccharide belonging to the group of fructans. In addition to a terminal glucose building block, the chain contains up to 60 fructose monomers, each of which is linked via β-2,1-glycosidic bonds. Inulin may be obtained from the leaves, roots, fruits and/or flowers of composites and/or umbellifers, such as Jerusalem artichokes, chicory, artichokes and/or parsnips.

Cationic inulin polymers particularly suitable as contemplated herein are cationically modified by reacting hydroxyl groups of the fructose building blocks with reactive quaternary ammonium compounds. Suitable quaternary ammonium compounds are preferably compounds of the following formula

N⁺(R¹R²R³R⁴)X⁻

in which R¹, R² and R³ are methyl or ethyl groups and R⁴ is an epoxy-R⁵— or a halohydrin group Y—CH₂—CH(OH)—R⁵—, in which R⁵ is a C₁-C₃ alkylene group, Y is a halide and X is an anion such as Cl—, Br—, I— or HSO4. Particularly suitable cationic inulin polymers b) for the purposes of the present disclosure correspond to the formula

R—O—CH₂—CH(OH)—R⁵—N⁺(R¹R²R³)X⁻,

wherein R is inulin and the other residues have the same meaning as above.

In a particularly preferred embodiment, the compositions as contemplated herein contain cationic inulin polymers cationically modified with cationic hydroxy-C₁-C₃-alkyl-trialkylammonium groups, in particular with hydroxypropyltrimethylammonium groups. Within this version, especially cationic inulin polymers known under the INCI designation Hydroxypropyltrimonium Inulin and commercially available (for example commercially available under the trade name Quatin 1280 TQ-D from Cosun Biobased Products) are preferred.

The degree of cationic substitution of cationic inulin polymers, especially of cationic inulins known under the INCI designation Hydroxypropyltrimonium Inulin, can be varied and adjusted as required. For use in the cosmetic compositions as contemplated herein, it has been shown to be particularly preferred when such cationically modified inulin polymers exhibit a higher degree of cationic modification (higher degree of cationic substitution), because this allows better coacervate formation and ultimately better care performance to be achieved in the compositions.

In a particularly preferred embodiment, the cationic inulin polymer contained in the cosmetic compositions as contemplated herein has a cationic charge density from about >1.5 meq/g, more preferably about >2.0 meq/g, particularly preferably about >2.5 meq/g, very preferably about >3.0 meq/g and especially about >3.5 meq/g. Within this version it is particularly preferred if cationic inulin polymers known under the INCI designation Hydroxypropyltrimonium Inulin b) have a cationic charge density from about >1.5 meq/g, more preferably about >2.0 meq/g, particularly preferably about >2.5 meq/g, very particularly preferably about >3.0 meq/g and particularly about >3.5 meq/g.

In another particularly preferred version, the cationic inulin polymer b) contained in the cosmetic compositions as contemplated herein has an average molar mass of from about 2,000 to about 50,000 g/mol, more preferably from about 2,500 to about 40,000 g/mol, particularly preferably from about 3,000 to about 30,000 g/mol, very preferably from about 3,500 to about 20,000 g/mol and in particular from about 4,000 to about 10,000 g/mol. Within this version it is particularly preferred if cationic inulin polymers known under the INCI designation Hydroxypropyltrimonium Inulin b) have an average molar mass of from about 2,000 to about 50,000 g/mol, more preferably from about 2,500 to about 40,000 g/mol, particularly preferably from about 3,000 to about 30,000 g/mol, very preferably from about 3,500 to about 20,000 g/mol and particularly from about 4,000 to about 10,000 g/mol.

The cationic inulin polymer(s)—preferably compounds known under the INCI designation Hydroxypropyltrimonium Inulin—are preferably used in the cosmetic cleansing compositions as contemplated herein in an amount of from about 0.01 to 5.00 wt.-%, more preferably from about 0.02 to about 4.00% by weight, particularly preferably from about 0.03 to about 3.00% by weight, very particularly preferably from about 0.04 to about 2.50% by weight and in particular from about 0.05 to about 2.00% by weight (based on the total weight of the cleansing compositions).

In a further preferred embodiment, the cosmetic compositions as contemplated herein contain, instead of the cationic inulin polymer or in addition to the cationic inulin polymer, at least one other cationic polymer selected from cationic polymers of natural origin, preferably from cationic polygalactomannan derivatives.

Galactomannans are polysaccharides including combinations of mannose and galactose monomers in different contents. In it, the mannose units are connected to each other via β(1-4)-glycosidic bonds; the galactose units via α(1-6)-bonds. The ratio of mannose to galactose monomers varies according to the type and origin of the plant and the temperature at which it was grown. In Greek fenugreek gum, the mannose-galactose ratio is about 1:1 (corresponding to one monomer of mannose to one monomer of galactose); in guar gum about 2:1; in tara gum about 3:1; in locust bean gum about 4:1 and in cassia gum about 5:1. All galactomannans from these sources are suitable for cationic modification and use as polymers in cosmetic compositions as contemplated herein. Guar gum and/or cassia gum are particularly suitable for use in cosmetic products as contemplated herein.

Like the cationic inulin polymers, the galactomannans, preferably galactomannans from the aforementioned sources, can be cationically modified by reacting the hydroxyl groups of the galactomannan polymers with reactive quaternary ammonium compounds. Suitable quaternary ammonium compounds are preferably compounds of the following formula

N⁺(R¹R²R³R⁴)X⁻

in which R¹, R² and R³ are methyl or ethyl groups and R⁴ is an epoxy-R⁵— or a halohydrin group Y—CH₂—CH(OH)—R⁵—, in which R⁵ is a C₁-C₃ alkylene group, Y is a halide and X is an anion such as Cl—, Br—, I— or HSO4. Particularly suitable cationic galactomannane polymers within the meaning of the present disclosure correspond to the formula

R—O—CH₂—CH(OH)—R⁵—N⁺(R¹R²R³)X⁻,

wherein R is the respective galactomannan and the other radicals have the same meaning as above.

In a particularly preferred embodiment, the compositions as contemplated herein therefore contain cationic galactomannan polymers cationically modified with cationic hydroxy-C₁-C₃-alkyl-trialkylammonium groups, in particular with hydroxypropyltrimethylammonium groups. Within this version, galactomannan polymers which have been cationically modified with cationic hydroxy-C₁-C₃-alkyl-trialkylammonium groups, in particular with hydroxypropyltrimethylammonium groups, and which are derived from guar gum and/or cassia gum are particularly preferred.

In In a particularly preferred form, the cosmetic compositions as contemplated herein contain, as a cationic polygalactomannan derivative, at least one of the compounds known under the INCI designations Guar Hydroxypropyltrimonium Chloride, Hydroxypropyl Guar Hydroxypropyltrimonium Chloride and/or Cassia Hydroxypropyltrimonium Chloride.

Guar and Cassia polymers known under these INCI designations are commercially available from various suppliers, for example under the designations Jaguar®, N-Hance®, Polycare®, Clearhance®, Activsoft®, Guarquat®, Vida-Care®. Jaguar® C-162, Jaguar® C500, Jaguar® Styl 100, N-Hance® 3196, N-Hance® HPCG 1000, Activsoft® C17, Guarquat® C130 KC, Guarquat® CP500 KC, Vida-Care® GHTC and/or Polycare® Split Therapy are specific examples of cationic polymers of natural origin that are particularly suitable as contemplated herein.

Cationic polymers suitable as contemplated herein are preferably used in the cosmetic compositions as contemplated herein (based on their total weight) in amounts of from about 0.01 to about 2.00% by weight, more preferably from about 0.02 to about 0.90% by weight, particularly preferably from about 0.03 to about 0.80% by weight, very particularly preferably from about 0.04 to about 0.70% by weight and in particular from about 0.05 to about 0.60% by weight.

The present disclosure further relates to a solid hair cosmetic composition as described above, further comprising—based on the total weight of the cosmetic composition—from about 0.01 to about 10.0% by weight of at least one oil, fat and/or wax component, preferably a naturally occurring oil, fat or wax.

These are caring substances that help to keep both the skin and hair structure healthy. The defined concentration range makes it possible to use this care effect but at the same time to exclude noticeable greasiness after application of an appropriate composition. Naturally occurring raw materials have the advantage that they grow again and can therefore be used sustainably. This aspect is also becoming increasingly important to many users.

It has been found that vegetable butters with a melting range of from about 20° C. to about 35° C. are particularly suitable for incorporation into cosmetic compositions as contemplated herein.

Accordingly, vegetable butters with a melting point in the range from about 20° C. to about 35° C., such as Shea butter (INCI designation), are particularly preferred: Butyrospermum Parkii (Shea) Butter), Mango Butter (INCI designation: Mangifera Indica (Mango) Seed Butter), Murumuru Butter (INCI designation: Astrocaryum Murumuru Seed Butter), cocoa butter (INCI designation: Theobroma Cacao (Cocoa) Seed Butter) and/or Cupuacu Butter (INCI designation: Theobroma Grandiflorum Seed Butter).

Cupuacu butter (INCI designation) is particularly preferred: Theobroma Grandiflorum Seed Butter) and/or Shea Butter (INCI designation: Butyrospermum Parkii (Shea) Butter) and especially preferred is Shea Butter (INCI designation: Butyrospermum Parkii (Shea) Butter).

The at least one vegetable butter (preferably Cupuacu butter and/or Shea butter; in particular shea butter) is used in the cosmetic compositions as contemplated herein preferably in a proportion by weight of from about 0.01 to about 10.00% by weight, more preferably of from about 0.05 to about 5% by weight, particularly preferably of from about 0.10 to about 1% by weight of the total weight of the compositions.

Oils suitable as contemplated herein are preferably perfume oils and/or vegetable triglyceride oils, such as coconut oil, (sweet) almond oil, walnut oil, peach kernel oil, apricot kernel oil, avocado oil, tea tree oil, soy bean oil, cotton seed oil, sesame oil, sunflower oil, tsubaki oil, evening primrose oil, rice bran oil, palm oil, Palm kernel oil, mango kernel oil, cranberry oil, sea buckthorn oil, meadow foam herb oil, thistle oil, macadamia nut oil, grape seed oil, amaranth seed oil, argan oil, bamboo oil, olive oil, Wheat germ oil, pumpkin seed oil, mallow oil, hazelnut oil, safflower oil, corn oil, olive oil, rapeseed oil, canola oil, sasanqua oil, jojoba oil, rambutan oil, manila oil and/or quinoa oil.

Particularly preferred are argan oil, jojoba oil, manila oil, macadamia nut oil, pumpkin seed oil, amaranth seed oil, quinoa oil, soy bean oil, cotton seed oil, sunflower oil, palm oil, palm kernel oil, linseed oil, almond oil, corn oil, olive oil, rapeseed oil, sesame oil, soy bean oil, thistle oil, wheat germ oil, peach kernel oil, cranberry oil, sea buckthorn oil and/or coconut oil.

Especially preferred are argan oil, jojoba oil, marula oil, almond oil, olive oil, coconut oil and/or sunflower oil.

The present disclosure further relates to a solid hair cosmetic composition as previously described, further comprising—based on the total weight of the cosmetic composition—from about 0.001 to about 0.01% by weight of at least one bittering agent, preferably a compound known under the INCI designation Denatonium Benzoate.

A bitter substance is particularly important in cosmetics, household products etc. which are made up in such a way that their shape, colour, feel etc. appeal to small children or babies and encourage them to play, although swallowing could also occur. A bitter substance prevents this. Denatonium benzoate is an extremely strong bittering agent and is therefore particularly effective even at exceptionally low application concentrations. Furthermore, it is not associated with any known adverse effects.

In addition to the ingredients described above, the cosmetic compositions as contemplated herein may contain at least one active ingredient advantageously selected from the group comprising plant extracts, humectants, protein hydrolysates, perfumes, UV filters, structurants such as maleic acid, dyes for colouring the composition, Active ingredients such as bisabolol and/or allantoin, antioxidants, preservatives such as sodium benzoate or salicylic acid, additional viscosity regulators such as salts (NaCl) or polymers, and pH adjusters such as α- and β-hydroxycarboxylic acids such as citric acid, lactic acid, malic acid, glycolic acid, and/or bases such as alkanolamines and/or sodium hydroxide).

Suitable plant extracts are extracts that can be produced from all parts of a plant. Usually these extracts are produced by extraction of the whole plant. However, in some cases it may be preferable to produce the extracts exclusively from flowers and/or leaves of the plant. Especially suitable are extracts from Paeonia Lactiflora, Rosa Damascena Flower, Malus Domestica Fruit, Argania Spinosa Shell Powder, Laminaria Saccharina, Cannabis Sativa, Green Tea, Oak bark, Nettle, Hamamelis, Hops, Chamomile, Burdock root, Horsetail, Hawthorn, Lime blossom, Litchi, Almond, Aloe Vera, Spruce needle, Horse chestnut, Sandalwood, juniper, coconut, mango, apricot, lime, wheat, kiwi, melon, orange, grapefruit, sage, rosemary, birch, mallow, cuckooflower, thyme, yarrow, Thyme, lemon balm, cowslip, marshmallow, ginseng, ginger root, Echinacea purpurea, Olea europea, Boerhavia diffusa roots, Foeniculum vulgaris and Apim graveolens. The extracts of Paeonia Lactiflora, Rosa Damascena Flower, Malus Domestica Fruit, Argania Spinosa Shell Powder, Laminaria Saccharina, Cannabis Sativa, Green Tea, Nettle, Hamamelis, Chamomile, Aloe Vera, Ginseng, Echinacea purpurea, Olea europea and/or Boerhavia Diffusa roots are particularly preferred for use in the compositions as contemplated herein. Water, alcohols and mixtures thereof may be used as extraction agents for the preparation of the above plant extracts. Among the alcohols, lower alcohols such as ethanol and isopropanol, but especially polyhydric alcohols such as ethylene glycol and propylene glycol, both as the sole extracting agent and mixed with water, are preferred. Plant extracts based on water/propylene glycol in a ratio of from about 1:10 to about 10:1 have proven to be particularly suitable. The plant extracts can be used both in pure and diluted form. If they are used in diluted form, they usually contain approx. from about 2-80% by weight of active substance and the extraction agent or mixture of extraction agents used in their extraction as solvent. The plant extracts can be used in the hair treatment compositions as contemplated herein (based on the total weight of the compositions) preferably in an amount of from about 0.01 to about 10% by weight, more preferably of from about 0.05 to about 7.5% by weight and especially of from about 0.1 to about 5% by weight.

Among suitable pH adjusting agents, particularly preferred for use in the compositions as contemplated herein are citric acid, lactic acid, malic acid, glycolic acid.

The present disclosure further relates to a solid hair cosmetic composition as described above for the cleansing and/or care of human skin and/or human hair.

With such a solid hair cosmetic composition the above described benefits can be achieved on human skin and/or human hair.

The present disclosure further relates to a solid hair cosmetic composition as described above in the form of a pen or piece.

These forms of packaging each have certain advantages. One piece still resembles in some ways traditionally used cosmetics and is often preferred by less experimental users. A stick is easier to transport because it is usually fitted with a cap or sleeve and is easier to hold because it can be grasped by its base, sleeve or outer packaging.

As can be seen from the production processes and applications revealed herein, the pen is well suited for multiple use by the user. The present disclosure therefore represents the solid hair cosmetic composition as described above in the form of a multiple use stick.

The piece can be dimensioned in such a way that it can be used either individually or multiple times. Particular preference is given to pieces that are sized to allow the piece to be used only once. The present disclosure therefore provides for the solid hair cosmetic composition as described above in the form of a disposable or reusable piece, preferably in the form of a disposable piece.

The present disclosure further relates to a solid hair cosmetic composition as described above, with a Shore A hardness value of from about 10 to about 30 and a Shore AO hardness value of from about 5 to about 60.

Such a hardness range is perceived as pleasant by users. In this area it is possible to take a small amount of solid hair cosmetic composition for one application from a jar intended for several applications with the hands and without any other aids.

If a Shore A hardness is indicated in the context of this application, it means that the hardness was determined by measurement with a needle with a truncated conical tip, the face of the truncated cone having a diameter of 1.3 millimetres at an angle of 35°. If a Shore AO hardness is specified, this was measured by measuring with a needle with a spherical segment-shaped rounded tip with a radius of the spherical segment of 2.5 mm One durometer was “set” on the product without additional pressure and the maximum value was read. One multiple measurement (at least 3 measurements) was performed in each case and the mean value was given. A manual analogue durometer from Sauter GmbH was used. Unless otherwise expressly described, the instructions enclosed with this device, version 1.2, dated August 2014, which states that it complies with DIN 53505, ASTM D2240 and ISO 868, have been followed.

The present disclosure further relates to a process for producing the solid hair cosmetic composition as described above, comprising the process
a) Mix all ingredients with sufficient heat and agitation to ensure that all ingredients are melted and/or evenly distributed,
b) Allow the resulting mixture to cool and solidify.

Such a process makes an evenly blended solid hair cosmetic composition as described above actually available and makes its benefits tangible.

The present disclosure further relates to a process for producing the solid hair cosmetic composition as described above, comprising the process:
a) Mix all ingredients with sufficient heat and agitation to ensure that all ingredients are melted and/or evenly distributed,
b) Pour the resulting mixture into a pin/piece mould,
c) Allow the mixture to cool and solidify.

Such a process makes an evenly blended solid hair cosmetic composition in stick or piece form as described above actually available and its related benefits described above can be experienced.

The present disclosure further relates to a process for producing the solid hair cosmetic composition as described above, comprising the process:
a) Mix all ingredients with sufficient heat and agitation to ensure that all ingredients are melted and/or evenly distributed,
b) Pour the resulting mixture into a mould,
c) Allow the mixture to cool and solidify.

Such a process makes an evenly blended solid hair cosmetic composition actually available in a specific as described above and makes it possible to experience its related benefits described above.

The present disclosure further relates to a method for cleaning hair in which a solid hair cosmetic composition is mixed with water as described above and applied to the hair or in which a stick or piece is applied directly to wet hair and worked in.

This procedure makes the advantages described above tangible for a user and thus represents an enrichment not only for personal hygiene and care but also a sensory enrichment.

The present disclosure further relates to a use of a solid hair cosmetic composition as described above for cleaning and/or care of the hair.

This use makes the advantages described above tangible for a user and thus represents an enrichment not only for personal hygiene and care but also a sensory enrichment.

As can be seen from these described designs and their advantages, the process and packaging aspects are important for the present disclosure. They are discussed in more detail below.

In a manufacturing process as contemplated herein, for example, all ingredients are placed in a heatable container, such as, on a laboratory scale, in a suitable vessel in a water bath or on a heating plate, on a production scale rather in a closed and pressurizable vessel, and are mixed and heated, in the recipes as contemplated herein, for example at about 75° C., until all ingredients are sufficiently mixed. In such a process, different temperature steps can also be run. For example, components that can be homogeneously mixed even at a relatively low temperature can be mixed first. This can happen at from about 40° C. to about 50° C. It can also be advantageous to mix in certain ingredients at higher temperatures, for example at from about 85° C. to about 90° C. For this purpose, a process as contemplated herein may comprise one or more steps in this temperature range. Afterwards, one or more steps can be carried out at a lower temperature again, in which further components are mixed in. Typically, the compositions as contemplated herein solidify at about 65° C., so that certain process steps, such as mixing and extruding the finished mixtures, are not reasonably possible below such a temperature level.

If a composition as contemplated herein is extruded, the available shape can be determined by a shape of the die closing the extruder. The solidifying mixture can be filled into moulds, portioned on a base or produced endlessly and cut and portioned at the nozzle or afterwards.

It is also important to note that cosmetic products have a lot to do with feeling, fun and emotions. Many people relax during personal hygiene and enjoy the pleasant feeling of doing something good for themselves. Especially since many people find their everyday life more and more demanding or stressful, small pleasures and playfulness are an important point at which stress can be released from a person and satisfaction can be created. Solid cosmetic preparations with incorporated gas phase, i.e. to a certain extent solid foams, feel different from conventional products, which is perceived as interesting and pleasant.

The compositions as contemplated herein are also suitable for some other forms of packaging not yet described in detail. For example, a stick or pen can be realized. One end of the pen may remain in a wrapper or package during use, so that a user does not have the problem of holding on to a slippery piece of solid cosmetic preparation. The design can be chosen, for example, like a shaving soap, where there is usually a fixed base on one side of the pen and the pen itself is surrounded by a sleeve that can be easily removed by hand, or it can be more similar to a deodorant stick, i.e. include a fixed sleeve also around the outer circumferential surface of the pen as well as a mechanism to gradually advance the pen inside the sleeve so that it always protrudes slightly beyond the opening until it is finally completely worn away by repeated use. The packaging for transport, for example during a journey, is also quite simple with a pen, as a cap surrounding or covering the pen can easily be attached. Pens would be problematic with a conventional soap formulation because the material removal on the relatively small surface would be too slow. With the compositions as contemplated herein, they can be easily realized and the speed of material removal during application meets the expectations of the users without, on the other hand, leading to wasteful use through excessive removal.

The extrusion processes described above can also be used to produce interesting shapes reminiscent of injection-moulded biscuits. Thanks to specially shaped dies on the extrusion die, a variety of shapes can be realized, for example a heart or clover shape. An extruded strand thus obtained can then be cut into pieces or slices, providing emotionally appealing small portions of the solid cosmetic compositions as contemplated herein. Similarly, it is possible to roll out an extruded strand or other form of a composition as contemplated herein and then, by punching or cutting, produce pieces that are similar to cookie cutters in terms of shaping properties.

It is also possible to pour the solid cosmetic compositions as contemplated herein into a crucible, for example a glass jar. Since the strength of these compositions is in a range that allows an application portion to be taken manually, without a tool, from a crucible intended for multiple applications. If the composition has been foamed in a crucible during its manufacture, the result is a particularly interesting feeling.

It is also possible to make up solid cosmetic compositions reminiscent of a piece of paper, a foil or a wafer, which brings with it a new and pleasant feeling during application. Since the thickness of the coating is small in this type of packaging, short dissolution times are possible, which accommodates impatient users and does not encourage wasteful use of water. A product packaged in this way may be placed on the market in a packaging unit in which a large number of leaves or flakes are placed in a small carton, possibly subdivided, so that a single withdrawal is possible.

After various designs and their respective advantages were explained in detail, the presentation of exemplary compositions and an exemplary manufacturing process follows.

Two detailed exemplary compositions are shown in the following tables 1 and 2:

TABLE 1
Example 1

| | | Active substances contained | |
|---|---|---|---|
| Group | Ingredients | therein (INCI) | % |
| 1 | Pureact WS Conc | Aqua (Water), Sodium Methyl | 11.15 |
| | | Cocoyl Taurate, Coconut | |
| | | Acid, Sodium Chloride | |
| 1 | City water | Aqua (Water) | 0.25 |
| 1 | Glycerine 99.5% | Glycerine | 41 |
| 1 | Sodium stearate | Sodium Stearate | 6 |
| | OP-100V | | |
| 1 | Citric acid, anhydrous, | Citric Acid | 2 |
| | fine granular | | |
| 2 | ISELUX | Sodium lauroyl methyl | 2 |
| | | isethionates, sodium | |
| | | methyl isethionates | |
| 2 | Maltodextrin DE15 | Maltodextrin | 1 |
| 3a | Pureact I-80P | Sodium Cocoyl Isethionate | 25.5 |
| 3b | Maisita 9040 | Zea Mays (Corn) Starch | 5 |
| 4 | Avocado oil, cold | Persea Gratissima | 2 |
| | pressed | (Avocado) Oil, Ascorbyl | |
| | | Palmitate | |
| 4 | Preservatives | Phenoxyethanol | 1 |
| 4 | Fragrance | Perfume (Fragrance) | 1 |
| 5 | Quatin 1280 TQ-D | Hydroxypropyl Trimonium | 1 |
| | | Inulin | |
| 4 | Bitrex 2.5% in water | Aqua (Water), Denatonium | <0.1 |
| | | Benzoate | |
| 6 | Sodium bicarbonate, | Sodium Bicarbonate fine | 1 |
| | fine | | |

The exemplary procedure was carried out as follows:

The composition shown in detail in Table 2 was produced as follows: The ingredients were used in the ratio shown in Table 2 above. Pureact WS Conc was heated in a drum to 40° C. to 50° C. to melt and mix. After mixing until homogeneity, the other ingredients of group 1 (see table 2) were added. It was mixed again until homogeneity and then the temperature was increased to 85° C. to 90° C. At this temperature, the ingredients of Group 2 (see Table 2) were added and mixed in until homogeneous. This was then repeated with the ingredients of Group 3 (3a and 3b, see Table 2). The ingredients of Group 4 (see Table 2) were homogeneously mixed together and also added to the previously prepared mixture and mixed in until homogeneity was achieved. For the next steps, the temperature was no longer actively maintained at 85° C. to 90° C. but it was only ensured that it did not drop to 70° C. or less. Then all ingredients of Group 5 (see Table 2) were added sequentially and mixed in until homogeneous and the ingredient of Group 6 (see Table 2) was added and quickly mixed in until homogeneous. Finally, the mixture was kept at a temperature above 70° C. for filling or packaging. Finally, the mixture was kept at a temperature above 70° C. for filling or packaging. The resulting mixture was then poured into a pin/piece mould. The mixture was then allowed to cool and solidify.

The composition produced in the above-mentioned quantities is particularly suitable for formulating the shampoo as a disposable form.

TABLE 2
Example 2

| | Active substances contained | |
|---|---|---|
| Ingredients | therein (INCI) | Wt % |
| Pureact WS Conc | Aqua (Water), Sodium Methyl | 11.15 |
| | Cocoyl Taurate, Coconut Acid, | |
| | Sodium Chloride | |
| City water | Aqua (Water) | 6.85 |
| Glycerine 99.5% | Glycerine | 41 |
| Sodium Stearate OP-100V | Sodium Stearate | 6 |
| Citric acid anhydrous | Citric Acid | 1.3 |
| fine granular | | |
| ISELUX | Sodium Lauroyl Methyl Isethionate | 2 |
| Agenamalt 20.225 | Maltodextrin | 1 |
| Maltodextrin DE15 | | |
| Pureact I-80P | Sodium Cocoyl Isethionate | 25.5 |
| Apricot Kernel Oil, | Prunus Armeniaca | 2 |
| cold pressed | (Apricot) Kernel Oil | |
| Phenoxyethanol, pure | Phenoxyethanol | 1 |
| Quatin 1280 TQ-D | Hydroxypropyl Trimonium Inulin | 1 |
| Crodarom Hemp NP/ | Aqua (Water), Glycerin, | 0.2 |
| NA 22880 | Cannabis Sativa Seed Extract | |
| Perfume Tea | Perfume (Fragrance) | 1 |
| Grandiosa 611084 | | |

The composition produced in the above-mentioned quantities is particularly suitable for formulating the shampoo as a multi-purpose form. Thus the process is similar to that described in example 1 above, but the resulting mixture is poured into a larger, appropriately sized mould.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Solid hair cosmetic composition comprising—based on the total weight of the cosmetic composition—
a) from about 30.0 to about 60.0% by weight of at least one polyhydric alcohol, and optionally:
b) from about 15.0 to about 50.0% by weight of at least one surfactant,
c) from about 0.1 to about 15.0.% by weight of at least one saturated or unsaturated, branched or unbranched C₈-C₃₀ alcohol and/or a saturated or unsaturated, branched or un-branched C₈-C₃₀ carboxylic acid and/or a salt of a saturated or unsaturated, branched or un-branched C₈-C₃₀ carboxylic acid, and
d) from about 0.1 to about 20.0% by weight of at least one polysaccharide.

2. Solid hair cosmetic composition according to claim 1, comprising glycerol as polyhydric alcohol a).

3. A solid hair cosmetic composition according to claim 1, comprising—based on the total weight of the cosmetic composition—
a) From about 30.0 to about 60.0% by weight of at least one polyhydric alcohol,
b) from about 15.0 to about 50.0% by weight of at least one surfactant,
c) from about 0.1 to about 15.0.% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20.0% by weight of at least one polysaccharide.

4. Solid hair cosmetic composition according to claim 1, comprising—based on the total weight of the cosmetic composition—
bi) from about 10.0 to about 40% by weight of at least one anionic isethionate surfactant and
bii) from about 1.0 to about 10% by weight of at least one anionic taurate surfactant.

5. A solid hair cosmetic composition according to claim 1, comprising as surfactant b)
i. at least one anionic isethionate surfactant of formula (I) 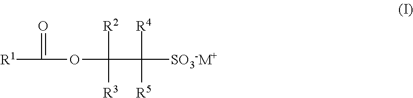
located in the
R¹ represents a linear or branched, saturated or unsaturated alkyl radical containing 6 to 30 carbon atoms,
R² to R⁵ independently of one another represent a hydrogen atom, a C₁-C₄ alkyl radical or a C₁-C₄ hydroxyalkyl radical, and
M⁺ represents an ammonium, an alkanol ammonium or a metal cation and/or
ii. at least one anionic taurate surfactant of formula (II) 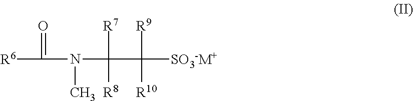
located in the
R⁶ represents a linear or branched, saturated or unsaturated alkyl radical containing 6 to 30 carbon atoms,
R⁷ to R¹⁰ independently of one another represent a hydrogen atom, a C₁-C₄ alkyl radical or a C₁-C₄ hydroxyalkyl radical, and
M⁺ stands for an ammonium, an alkanol ammonium or a metal cation

6. Solid hair cosmetic composition according to claim 1, comprising as component c) saturated or unsaturated, branched or unbranched C₈-C₃₀-carboxylic acids and/or their salts, C₁₀-C₂₂-carboxylic acids and/or their salts and in particular coco acids, lauric acid, myristic acid, palmitic acid, stearic acid, behenic acid, oleic acid and mixtures thereof and/or the salts of these acids.

7. A solid hair cosmetic composition according to claim 1, comprising as component c) coco acids, lauric acid, myristic acid, palmitic acid, stearic acid, behenic acid, oleic acid and mixtures thereof and/or the salts of these acids.

8. A solid hair cosmetic composition according to claim 1, comprising as component c) lauric acid, myristic acid, palmitic acid, stearic acid, as well as mixtures thereof and/or the salts of these acids.

9. A solid hair cosmetic composition according to claim 1, comprising as component c) sodium palmitate and/or sodium stearate.

10. Solid hair cosmetic composition according to claim 1, comprising as polysaccharide d) starch and/or modified starch and/or dextrins.

11. Solid hair cosmetic composition according to claim 1 in the form of a pen or piece.

12. Solid hair cosmetic composition according to claim 1, with a Shore A hardness value of from about10 to about 30 or a Shore AO hardness value of from about 5 to about 60.

13. A process for preparing the solid hair cosmetic composition according to claim 1, comprising:
a) Mixing all ingredients with sufficient heat and agitation to ensure that all ingredients are melted and/or evenly distributed,
b) Allowing the resulting mixture to cool and solidify.

14. A process for preparing the solid hair cosmetic composition according to claim 1, comprising:
a) Mixing all ingredients with sufficient heat and agitation to ensure that all ingredients are melted and/or evenly distributed,
b) Pouring the resulting mixture into a pin/piece mould,
c) Allow the mixture to cool and solidify.

15. A method for cleansing hair, in which a solid hair cosmetic composition according to claim 1 is mixed with water and applied to the hair or in which a stick or piece is applied directly to wet hair and worked in.
